(19) United States
(12) Patent Application Publication
(10) Pub. No.: US 2021/0254170 A1
(43) Pub. Date: Aug. 19, 2021

(54) SYSTEM AND METHOD FOR CANCER PROGNOSIS

(71) Applicant: ILLUMINA, INC., San Diego, CA (US)

(72) Inventors: Mahdi Golkaram, San Diego, CA (US); Shile Zhang, San Diego, CA (US); Li Liu, San Diego, CA (US); Aaron Wise, San Diego, CA (US); Joyee Yao, San Diego, CA (US); Shannon Kaplan, San Diego, CA (US); Alex So, San Diego, CA (US); Michael Salmans, San Diego, CA (US); Raakhee Vijayaraghavan, San Diego, CA (US)

(21) Appl. No.: 17/172,380

(22) Filed: Feb. 10, 2021

Related U.S. Application Data
(60) Provisional application No. 62/977,010, filed on Feb. 14, 2020.

(51) Int. Cl. C12Q 1/6886

(52) U.S. Cl. CPC C12Q 1/6886; C12Q 2600/158; C12Q 2600/118; C12Q 2600/106

(57) ABSTRACT

A potential prognostic biomarker is based on a combination of a composite score generated by sequence analysis of global human endogenous retrovirus (hERV)/retro-transposon transactivation and a cell signature generated using deconvolution of immune cells within a tumor sample for predicting the efficacy of chemotherapeutic agents and immune checkpoint inhibitors. Correlation analysis of the composite score with cell signature within a tumor sample enables survival analysis in individuals receiving chemotherapeutic agents and immune checkpoint inhibitors.

### PRIORITY AND CROSS-REFERENCE TO RELATED APPLICATION

This application claims the benefit of U.S. Provisional Application 62/977010 filed on Feb. 14, 2020, which is hereby incorporated by reference in its entirety.

### BACKGROUND

### Field

The present disclosure is related to a prognostic biomarker for stratifying individuals suffering from cancer to determine which individuals are more likely to have severe forms of the disease. In particular, embodiments relate to a test that analyzes multiple markers that are predictive of the severity of tumors in individuals.

### Description of the Related Art

There is a need for new diagnostic and prognostic tests to help medical professionals understand the severity of cancer in individuals. In some cases, there are no effective ways to determine how severe the effects of a particular tumor may be on an individual. In addition, there is a need to develop robust tests for determining how successful a particular treatment has been for an individual.

### SUMMARY

In some embodiments, a method of predicting the outcome of treating an individual suffering from cancer with an anti-cancer treatment is provided.

In some embodiments, the method of predicting comprises obtaining RNA sequence expression data from a biopsy taken from an individual having cancer, analyzing the RNA sequence expression data to determine if expression of cell-type specific markers in the biopsy are above a threshold value, analyzing the RNA sequence expression data to determine if hERV/retro-transposon gene expression is found within the biopsy, determining the cancer prognosis of the individual based on the threshold value and presence of the hERV/retro-transposon gene expression in the biopsy, and combining the expression profile of cell-type specific markers and expression profile of hERV/retro-transposon transactivation antigens to predict an outcome of an anti-cancer treatment.

In some embodiments of the method of predicting, the cancer comprises a tumor and the biopsy is a tumor biopsy.

In some embodiments of the method of predicting, analyzing the RNA sequence expression data comprises performing a transcriptome sequence analysis of global human endogenous retrovirus (hERV)/retro-transposon transactivation.

In some embodiments of the method of predicting, obtaining RNA sequence expression data comprises isolating total RNA from the cells, and performing next generation sequencing on the RNA sample to obtain the RNA sequence expression data.

In some embodiments of the method of predicting, analyzing the RNA sequence expression data to determine if hERV/retro-transposon gene expression is found comprises measuring expression of the hERV 2650 gene located on chromosome 7.

In some embodiments of the method of predicting, the cancer is selected from the group consisting of colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

In some embodiments of the method of predicting, the anti-cancer treatment is selected from the group consisting of surgery, radiation therapy, chemotherapy, immunotherapy, targeted therapy, hormone therapy, stem cell transplant, cytokine therapy, gene therapy, cell therapy, phototherapy, thermotherapy, and sound therapy.

In some embodiments of the method of predicting, the anti-cancer treatment comprises an anti-cancer chemotherapeutic selected from the group consisting of Cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

In some embodiments of the method of predicting, the cell-type specific markers are selected from the group consisting of: human endogenous retroviral (HERV) gene expression markers, tumor infiltrating lymphocyte (TIL) markers, microsatellite instability (MSI) status markers, and tumor mutational burden (TMB) markers.

In some embodiments of the method of predicting, the cell-type specific markers comprise markers associated with one or more of CD8+ T, CD4+ T, and CD19+ B cells.

In some embodiments of the method of predicting, the hERV/retro-transposon gene expression level is calculated using a univariate analysis of hERV gene expression.

In some embodiments, a method of obtaining a cellular signature of cells infiltrating a tumor is provided.

In some embodiments, the method of obtaining a cellular signature comprises obtaining a tumor, isolating cells of the tumor, isolating total RNA from the cells, performing RNAseq to obtain RNA sequence expression data, analyzing the RNA sequence expression data using a deconvolution algorithm to obtain an expression profile of cell-type specific markers, and determining a fraction of a cell-type based on the expression profile of cell-type specific markers in the RNA sequence expression data.

In some embodiments, the method of obtaining a cellular signature further comprises comparing the expression profile of cell-type specific markers and/or the expression profile of hERV/retro-transposon transactivation antigens and/or the fraction of one or more immune cell types in the tumor to a predetermined threshold, and administering an immune checkpoint inhibitor therapy to a patient if the tumor obtained from said patient exhibits a fraction above the predetermined threshold.

In some embodiments of the method of obtaining a cellular signature, the cell-type specific markers comprise markers associated with one or more of CD8+ T, CD4+ T, and CD19+ B cells.

In some embodiments of the method of obtaining a cellular signature, the immune checkpoint inhibitor therapy comprises a checkpoint inhibitor selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments, a method of obtaining a composite score of global human endogenous retrovirus (hERV)/retro-transposon transactivation is provided.

In some embodiments, the method of obtaining a composite score method comprises obtaining a tumor, isolating cells of the tumor, isolating total RNA from the cells, performing RNAseq to obtain RNA sequence expression data, and analyzing the RNA sequence expression data to obtain an expression profile of hERV/retro-transposon transactivation antigens.

In some embodiments, the method of obtaining a composite score further comprises comparing the expression profile of cell-type specific markers and/or the expression profile of hERV/retro-transposon transactivation antigens and/or the fraction of one or more immune cell types in the tumor to a predetermined threshold, and administering an immune checkpoint inhibitor therapy to a patient if the tumor obtained from said patient exhibits a fraction above the predetermined threshold.

In some embodiments of the method of obtaining a composite score, the immune checkpoint inhibitor therapy comprises a checkpoint inhibitor selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

### BRIEF DESCRIPTION OF THE DRAWINGS

FIG. 1 shows a schematic of an embodiment of a tumor immune cell deconvolution process where the proportion of CD19, CD4 and CD8 positive immune cells are determined for a tumor sample.

FIG. 2A shows a line graph of data from a tumor immune cell deconvolution process based on a titration experiment in which RNA from various immune cells are spiked into RNA from a tumor sample.

FIG. 2B shows an embodiment of a 3-dimensional Principle Component Analysis (PCA) representation of purified immune cells and background samples before (left) and after (right) Fractional Recovery of Immune Cell Types In Oncology NGS (FRICTION) gene selection.

FIG. 2C shows an embodiment of a deconvolution of five melanoma samples. The ‘actual concentration’ from FACS sorting is plotted against the ‘predicted concentration’ of the FRICTION algorithm. R² values are reported for each cell type.

FIG. 2D shows an embodiment of deconvolution data of primary immune cells titrated into melanocyte cell background. Panel A shows transcripts per million (TPM). Panel B shows data for CDMini. Panel C shows data for LM22 and panel D shows data for Xgb. LM22 and CDMine are different sets of genes available in the literature. Xgb is obtained using a machine learning approach that automatically selected a subset of genes that are the most informative in deconvolving immune cells.

FIG. 2E shows an embodiment of deconvolution data of RNA from purified immune cells titrated into RNA from total tissue. Panel A shows transcripts per million (TPM). Panel B shows data for CDMini. Panel C shows data for LM22 and panel D shows data for Xgb.

FIG. 2F shows additional embodiments of examples of titration versus score for different cell types, including liver, lung, thyroid, esophagus and bladder.

FIG. 2G shows additional embodiments of examples of titration versus score with technical replicates from ovary, pancreas, kidney and rectum.

FIG. 3 shows a schematic representation of a retroviral DNA integration into a genome.

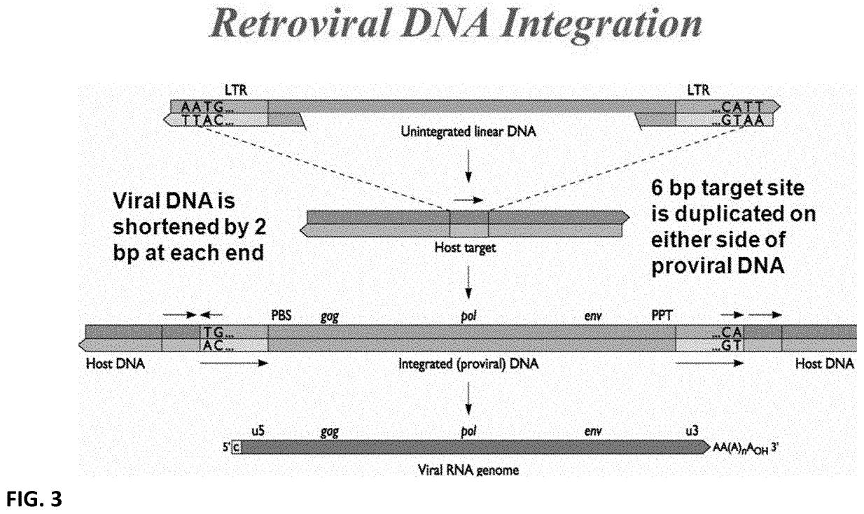

FIG. 4 shows schematic representation of expression of neoantigens in a tumor cell.

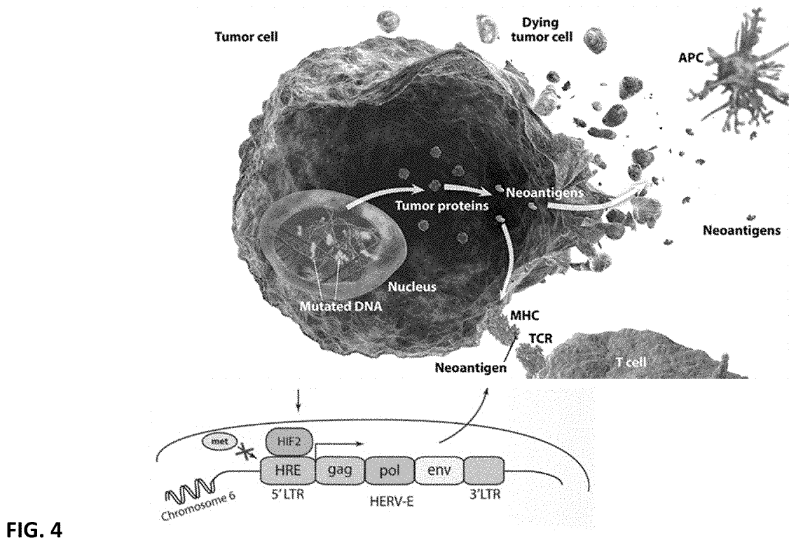

FIG. 5 shows a schematic of the prevalence of different viral antigens in various tumor types.

FIG. 6 shows a heat map of the association between Whole Exome Sequencing (WES) and Whole Transcriptome Sequencing (WTS) correlates.

FIG. 7 shows a bar graph of the frequency distribution of different hERV values and the median frequency (dotted line) of hERV in an individual sample.

FIG. 8 shows a bar graph of the frequency distribution of tumor infiltrating CD8+ T cells and the median frequency (dotted line) of tumor infiltrating CD8+ T cells in a tumor cell population.

FIG. 9 shows a bar graph of the frequency distribution of tumor infiltrating CD4+ T cells and the median frequency (dotted line) of tumor infiltrating CD4+ T cells in a tumor cell population.

FIG. 10 shows a bar graph of the frequency distribution of tumor infiltrating CD19+ T cells and the median frequency (dotted line) of tumor infiltrating CD19+ T cells in a tumor cell population.

FIG. 11 shows a line graph of an embodiment of cumulative high or low median survival data of a individual population based on hERV frequency. Below the line graph is listed the number of individuals in each group from each overall survival time point.

FIG. 12 shows a line graph of an embodiment of cumulative high or low median survival data based on hERV frequency. Below the line graph is listed the number of individuals in each group from each overall survival time point.

FIG. 13 shows a graph of an embodiment of cumulative high or low median survival data based on frequency of hERV 2650 located on chromosome 7. The numbers on the bottom show the number of individuals at the different time points.

FIG. 14 shows a graph of an embodiment of cumulative high or low median survival data based on frequency of hERV 2650 located on chromosome 7. The numbers on the bottom show the number of individuals at the different time points.

FIG. 15 shows a graph of correlation between the hazard ratio and frequency of the type of hERV.

FIG. 16 shows a line graph of an embodiment of overall survival data based on hERV and CD8 status of a tumor. The numbers on the bottom show the number of individuals at the different time points.

FIG. 17 shows a line graph of an embodiment of relapse free survival data of individuals based on hERV and CD8 status of a tumor in the individuals. The numbers on the bottom show the number of individuals at the different time points.

FIG. 18 shows a line graph of an embodiment of overall individual survival data based on clinicopathological status of individuals. The numbers on the bottom show the number of individuals at the different time points.

FIG. 19 shows a line graph of an embodiment of overall individual survival data based on clinicopathological status of the individual. The numbers on the bottom show the number of individuals at the different time points.

FIG. 20 shows a graph of an embodiment of overall individual survival data based on clinicopathological and WTS status of the individual. The numbers on the bottom show the number of individuals at the different time points.

FIG. 21 shows a graph of an embodiment of overall individual survival data based on clinicopathological and WTS status. The numbers on the bottom show the number of individuals at the different time points.

### DETAILED DESCRIPTION

Embodiments of the present disclosure relate to prognostic systems and methods for predicting the future health of an individual or individual. Embodiments relate to the discovery that the composite score generated from a transcriptome sequence analysis of global human endogenous retrovirus (hERV)/retro-transposon transactivation combined with a cell signature generated using deconvolution of immune cells within a tumor sample could be prognostic for predicting the health of an individual. In addition, the composite score may be useful for predicting the efficacy of chemotherapeutic agents and immune checkpoint inhibitors used on the individual population. In some embodiments, provided herein are survival analyses in individuals receiving chemotherapeutic agents and immune checkpoint inhibitors based on the composite score that is based on the level of hERV viral DNA and immune cell infiltration found in an individual's tumor sample.

There is a need for new diagnostic and prognostic biomarkers for cancers. For example, colorectal cancer (CRC) individuals have poor prognosis and there is a need for new diagnostic and prognostic biomarkers to avoid CRC-related deaths and avoid overtreatment. While the clinicopathological features such as tumor-node-metastasis (TNM) staging status at diagnosis, lymph node (LN) involvement (pNO-pN2), age, sidedness, etc. are well-established biomarkers of poor prognosis, the significance of molecular and cellular markers is well demonstrated in a clinical setting.

Some embodiments herein are related to methods of stratifying individuals to better predict the outcome of a treatment. In some embodiments, the treatment is an anti-cancer treatment. In some embodiments, the anti-cancer treatment is based on anti-cancer chemotherapeutics and/or checkpoint inhibitors. In some embodiments, the anti-cancer treatment is based on checkpoint inhibitors. In some embodiments, the anti-cancer treatment is based on anti-cancer chemotherapeutics or checkpoint inhibitors. In some embodiments, the anti-cancer treatment is based on anti-cancer chemotherapeutics and/or checkpoint inhibitors.

Non-limiting examples of anti-cancer chemotherapeutics include Cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

Non-limiting examples of checkpoint inhibitors include Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments, the systems and methods provided herein can be used to stratify individuals undergoing other forms of anti-cancer therapies. Non-limiting examples include surgery, radiation therapy, chemotherapy, immunotherapy, targeted therapy, hormone therapy, stem cell transplant, cytokine therapy, gene therapy, cell therapy, phototherapy, thermotherapy, and sound therapy.

### Deconvolution Analysis

In some embodiments, developing the composite score includes a method of obtaining a cellular signature of immune cells infiltrating a tumor. In some embodiments, the method comprises obtaining a tumor from an individual biopsy, isolating cells of the tumor, isolating total RNA from the cells, and performing next generation sequencing on the RNA sample (RNAseq) to obtain RNA sequence expression data for the transcriptome of the tumor cells. This tumor transcriptome is then analyzed using a deconvolution algorithm to obtain an expression profile of immune cell-type specific markers, and then determining a fraction of cells in the tumor sample based on the expression profile of cell-type specific markers in the RNA sequence expression data. A non-limiting example of a deconvolution analysis is provided in Example 1.

There is value in understanding the tumor microenvironment for its impact on tumor progression and immunotherapy efficacy. Computational tools based on gene expression data have shown promise for their ability to deconvolve the tumor microenvironment and report the types of immune cells present in heterogeneous tumor samples. In some embodiments, the method is related to obtaining a signature of cells infiltrating a tumor. In some embodiments, this information is used to determine the level of immune cell infiltration within a tumor. In some embodiments, this information is used to determine the type of cells that have infiltrated a tumor. In some embodiments, this information is used to determine the type of cells and the amount of each type of cell that have infiltrated a tumor.

Non-limiting examples of tumor/cancer include breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, or any neoplasm associated with brain including, but not limited to, astrocytomas (e.g., pilocytic astrocytoma, diffuse astrocytoma, anaplastic astrocytoma, and brain stem gliomas), glioblastomas (e.g., glioblastomas multiforme), meningioma, other gliomas (e.g., ependymomas, oligodendrogliomas, and mixed gliomas), and other brain tumors (e.g., pituitary tumors, craniopharyngiomas, germ cell tumors, pineal region tumors, medulloblastomas, and primary CNS lymphomas). In some embodiments, the tumor/cancer is related to one or more types of tumor/cancer provided herein.

The rise of immunotherapy in cancer treatment has resulted in increased interest in the immune microenvironment of the tumor. Better understanding the immune microenvironment could elucidate how and when immunotherapy will be effective.

A common approach to recapitulating the immune microenvironment is through cell type deconvolution, which models the complex mixture of cell types in a bulk tumor sample as a linear combination of a set of (characterized) prototypical cell signatures.

Without being limited by any particular theory, the high association between CD8+ T cells, Treg and PD1 indicates exhaustion of CD8+ T cells in most individuals. In some embodiments, tumor purity and immune infiltration are anti-correlated.

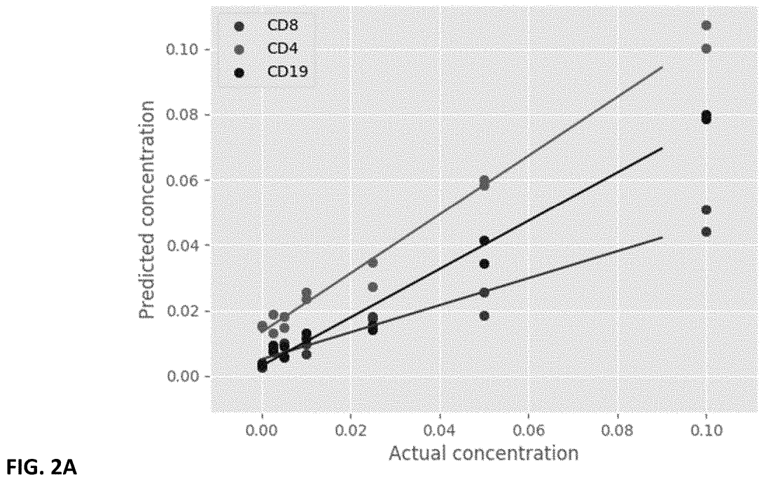

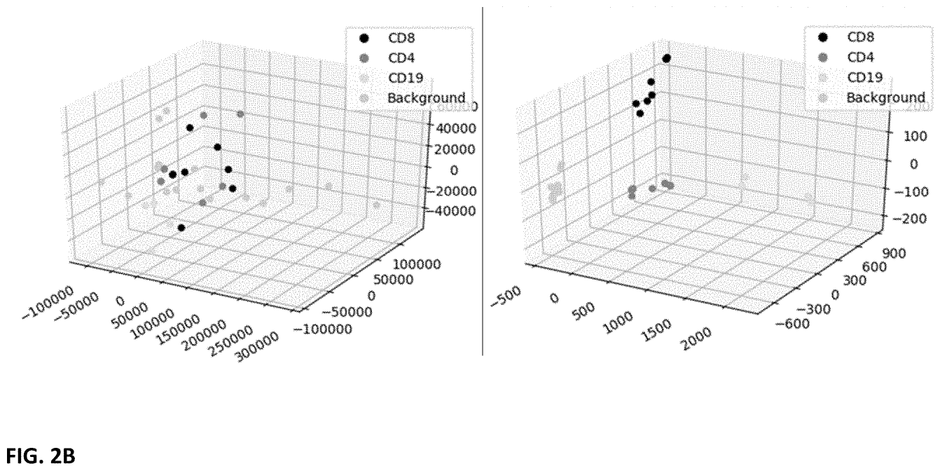

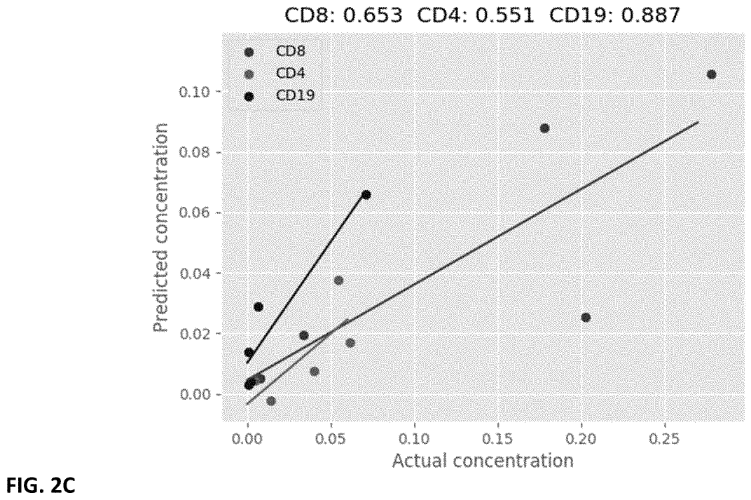

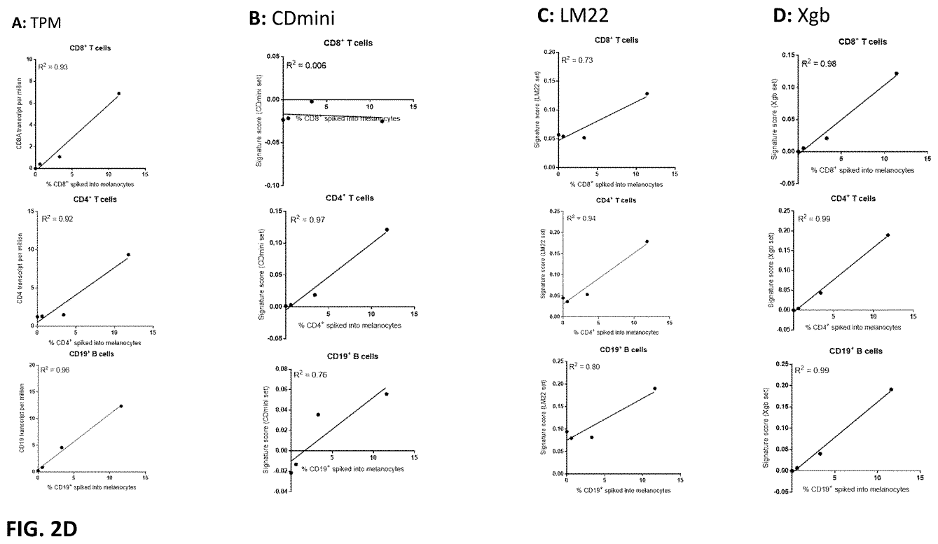

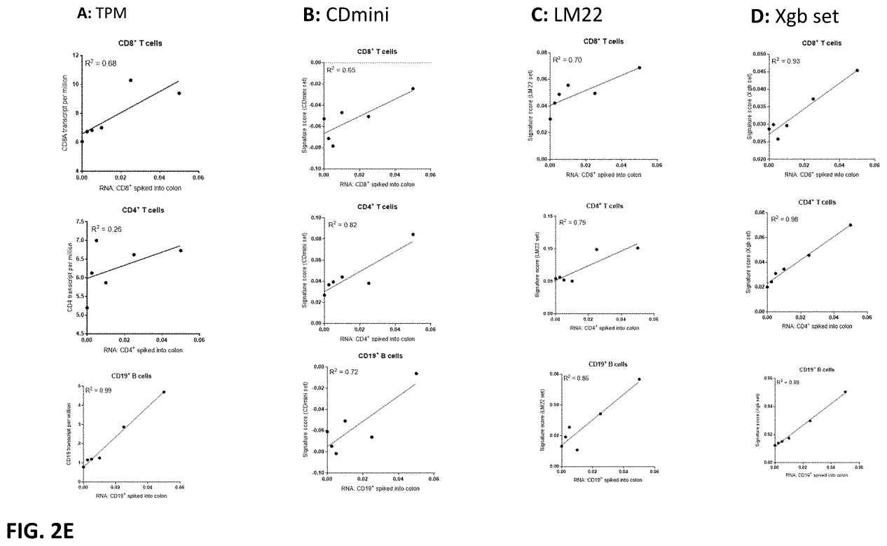

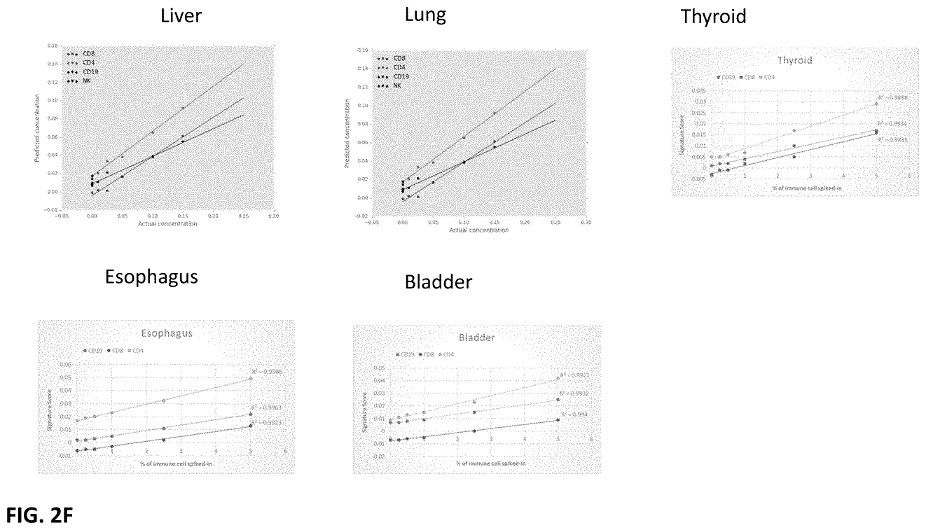

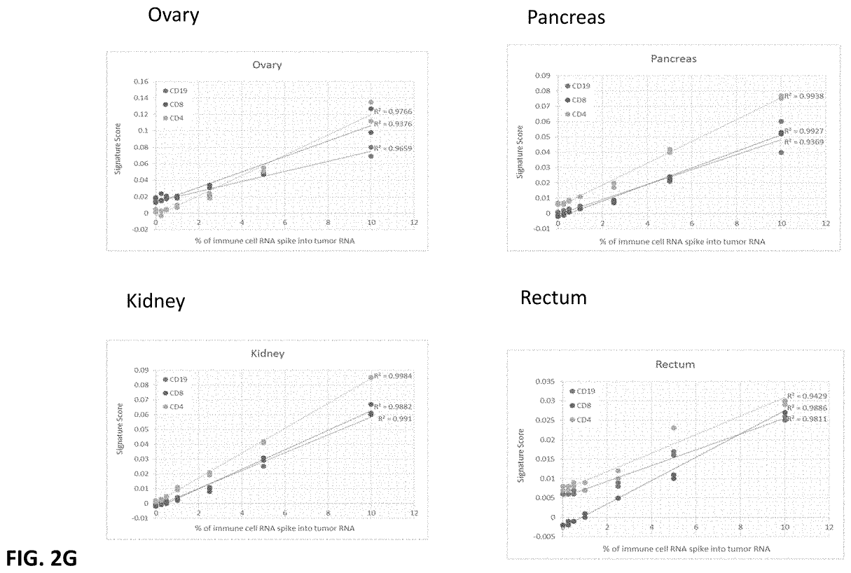

In some embodiments, a process termed “FRICTION”, for cell type deconvolution is provided (Example 1; FIGS. 2A-2C). While many state of the art deconvolution approaches report relative fractions or statistical enrichment, these embodiments focus on the careful selection and normalization of genes to better detect the absolute fractional level of cell types. To enable this, we performed a novel gene selection method that combined both statistical properties of gene expression along with the expression's ability to classify different cell types. Furthermore, we normalized against expression levels from over ten different control tissues to ensure robustness in many tissue backgrounds. FRICTION combines the gene selection and normalization techniques with a support vector regression based approach to deconvolution.

In some embodiments, FRICTION was trained to detect three cell types: CD8+ T, CD4+ T and CD19+ B cells. In some embodiments, the technique was validated using spike-in cell titrations, immunohistochemistry (IHC) staining of formalin-fixed, paraffin-embedded (FFPE) tumor samples and flow cytometry. The titration experiments (e.g., FIG. 2A in which RNA from carious immune cells are spiked into RNA from a tumor sample) demonstrate our method's linearity in a variety of tissue backgrounds (median R²>0.97), with high reproducibility among both technical and biological replicates and flow cytometry experiments demonstrate that this result extends to tumor samples.

In some embodiments, the FRICTION process provides a novel approach to cell type deconvolution, focusing on robust normalization and background correction to produce estimates of the absolute concentration of immune cell types. In some embodiments, FRICTION has been developed to be robust to many different tissue backgrounds, and produces an estimate of the fraction of each of its signature immune cell types. Tumors with immune cell infiltration can be candidates for anti-cancer chemotherapy, for example, using checkpoint inhibitors. Without being limited by any particular theory, it is believed that checkpoint inhibitors stimulate the immune system to generate an immune response against tumor antigens. In some embodiments, increased levels of immune cell infiltration are associated with increased patient response to checkpoint inhibitor therapy, and thus can be used as a biomarker to identify patients that are candidates for checkpoint inhibitor therapy. In some embodiments, immune cell infiltration above a threshold level is associated with increased responsiveness to checkpoint inhibitor therapy.

Though the current version of FRICTION is trained using signatures from three cell types (CD8+ T cells, CD4+ T cells and CD19+ B cells) the procedure is general such that, with gene expression data from additional cell types, further signatures could be added. Thus, in some embodiments, cell type deconvolution from RNA-seq data is possible. Non-limiting examples of other cellular signatures include B Cells, Dendritic Cells, Granulocytes, Innate Lymphoid Cells, Megakaryocytes, Monocytes/Macrophages, Myeloid-derived Suppressor Cells, Natural Killer Cells, Platelets, Red Blood Cells, T Cells, and Thymocytes. Other ongoing work involves further validation of the algorithm with additional flow cytometry experiments. Further algorithmic improvements to address correlated cell types and data normalization will continue to increase performance.

In some embodiments, the deconvolution analysis can be applied to other types of sequence data, for example, ATAC-seq data, which is generated by cutting accessible DNA and reading cluster around open chromatin. In some embodiments, ATAC-seq is quick and easy and may even be a more direct measure of cell type than RNA.

Human Endogenous Retrovirus (hERV) Analysis

In some embodiments, the composite score is generated by also including a method of obtaining a score of global human endogenous retrovirus (hERV)/retro-transposon transactivation. In some embodiments, the method is related to obtaining a score of transactivation of all hERV sequences in the genome. In some embodiments, the method comprises obtaining a tumor, isolating cells of the tumor, isolating total RNA from the cells, performing RNAseq to obtain RNA sequence expression data, and analyzing the RNA sequence expression data to obtain an expression profile of hERV/retro-transposon transactivation antigens.

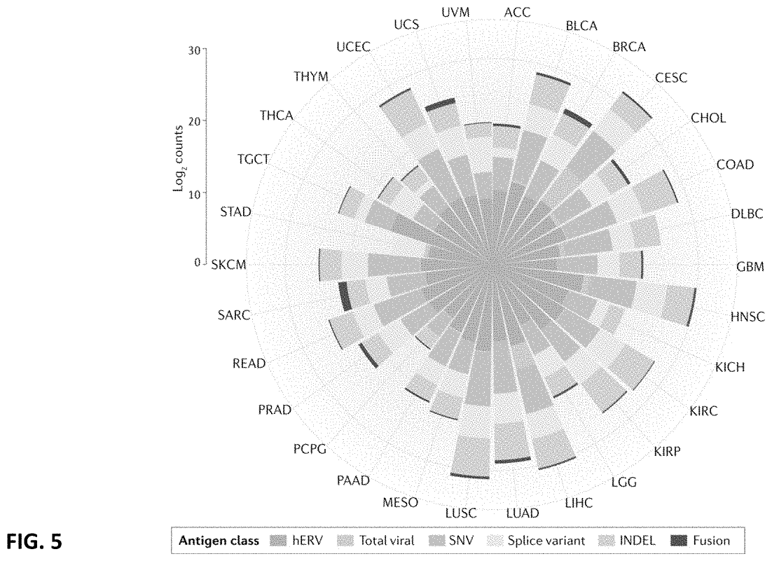

Viral sequences such as endogenous retrovirus (hERV) and/or retro-transposons are embedded in a genome. Normally, these viral sequences are silenced by methylation. However, in some tumors these silenced viral sequences are reactivated. Tumors with activated viral sequences can be candidates for anti-cancer chemotherapy, for example, using checkpoint inhibitors. Without being limited by any particular theory, it is believed that checkpoint inhibitors stimulate the immune system to generate an immune response against these viral sequences. FIG. 5 shows an embodiment of a schematic of the prevalence and frequency of different viral antigen sequences in various tumor types. hERV is the most common and has the highest frequency across different tumor types.

In some embodiments, a method of predicting how well an anti-cancer treatment may work on an individual is provided. In some embodiments, the method comprises obtaining RNA sequence expression data from a tumor biopsy taken from an individual having cancer, analyzing the RNA sequence expression data to determine if expression of immune cell markers in the tumor biopsy are above a threshold value, analyzing the RNA sequence expression data to determine if hERV/retro-transposon genes are found within the tumor biopsy, and determining if the cancer prognosis of the individual based on the threshold value and presence of the hERV/retro-transposon in the tumor biopsy.

As used herein, a “threshold” value is based on a percentile score. The threshold can vary based on an embodiment of the method or the parameter that is analyzed (e.g. hERV versus an immune cell). The threshold can also vary depending on the number of additional parameters included in an embodiment of a method.

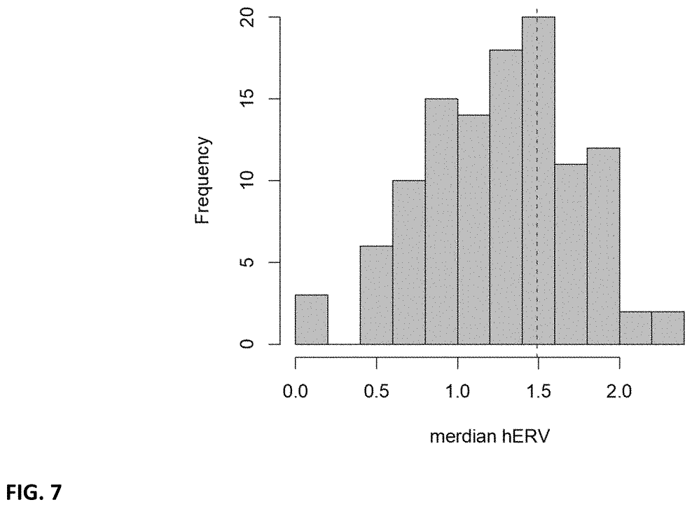

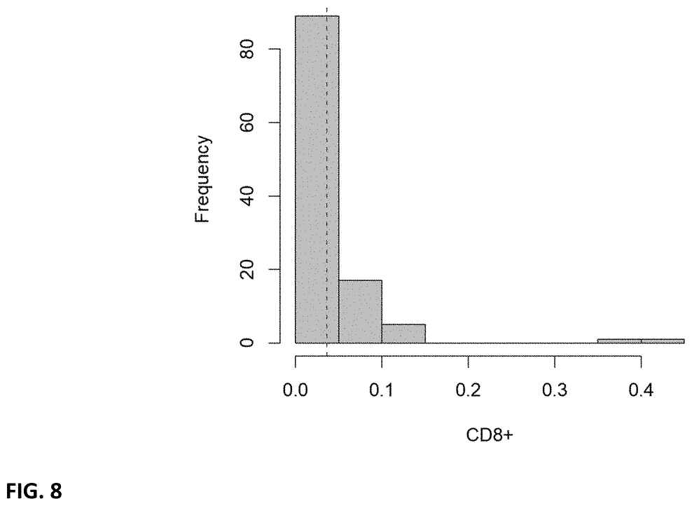

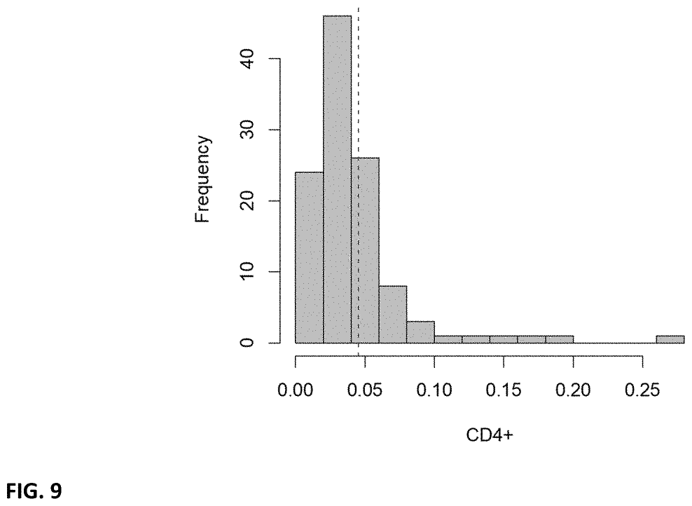

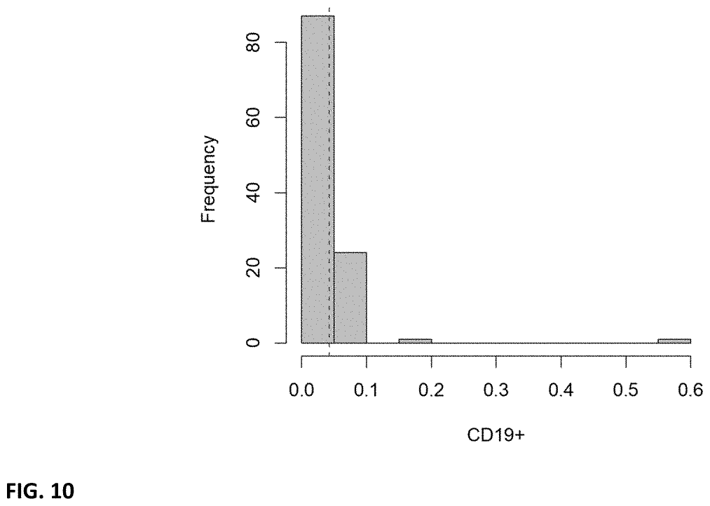

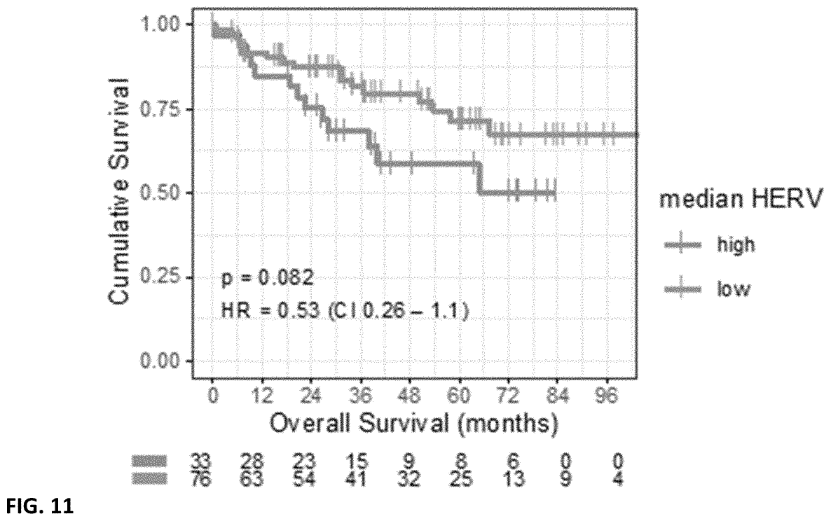

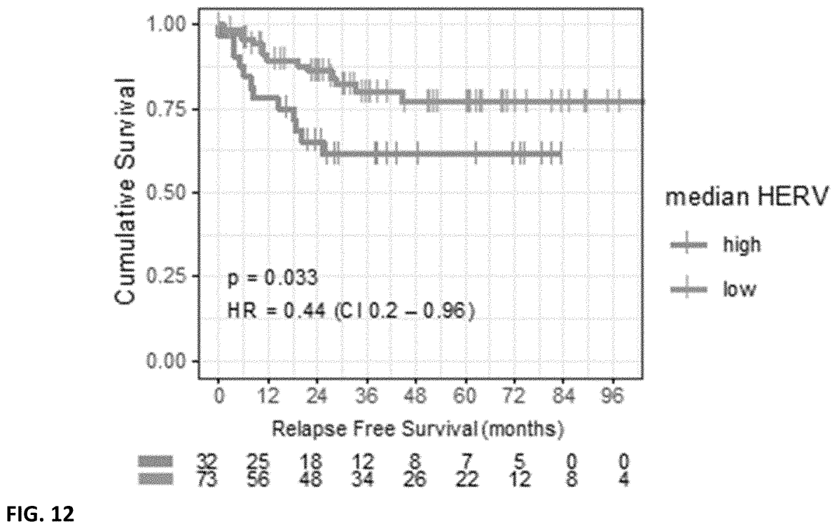

FIGS. 7-10 show the median frequency (dotted line) as thresholds for stratifying individuals into those with good or poor prognosis. In some embodiments, a score of transactivation of all hERV sequences is obtained (median hERV) and used to predict an outcome of anti-cancer treatment and overall survival or relapse free survival. As shown in FIGS. 11 and 12, a univariate analysis of hERVs only classified the individuals into those with good or poor prognosis. In some embodiments, a high median hERV correlated with poor prognosis in terms of overall survival (FIG. 11) as well as in terms of relapse free survival (FIG. 12). Overall survival (OS) is defined as the survival time from the date of diagnosis until the cut-off date. The cut-off date is the study end point date, which may be due to death or relapse or the last study follow-up. Relapse free survival (RFS) is defined as survival time from the date of surgery to the cut-off date. In contrast, in some embodiments, a low median hERV correlated with good prognosis in terms of overall survival (FIG. 11) as well as in terms of relapse free survival (FIG. 12).

The prognosis can be quantified using “hazard ratio” (HR), which is a probability of a “hazard” to a population (e.g., disease, debilitation, death, unresponsiveness to a treatment, etc.) determined as a statistics-based correlation between frequency to or more parameters (e.g., the type of hERV and one or more additional parameters as provided herein). For example, in the context of prognosis of responsiveness to an anti-cancer/tumor treatment, a lower hazard ratio would indicate a positive prognosis of response to treatment, and a higher hazard ratio would indicate a negative prognosis of response to treatment.

In some embodiments, the stratification of individuals based on a univariate analysis of hERVs only does not depend on the cancer type. In some embodiments, HR based on median hERV was universally applicable.

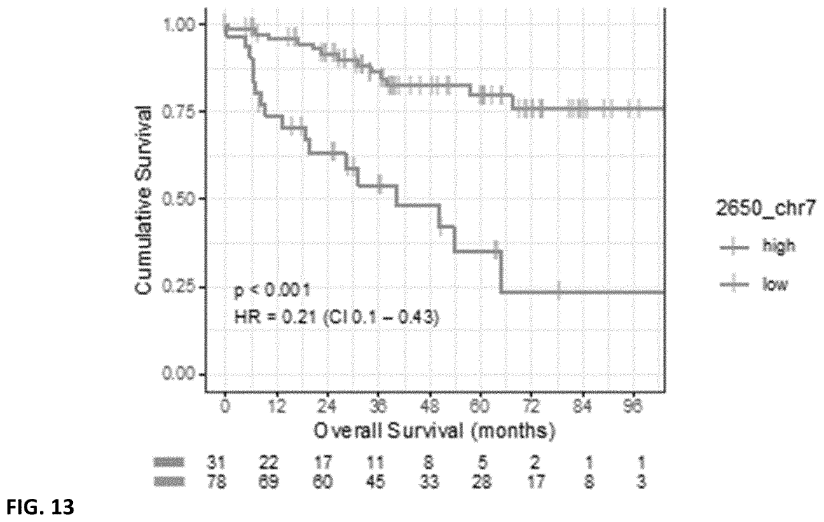

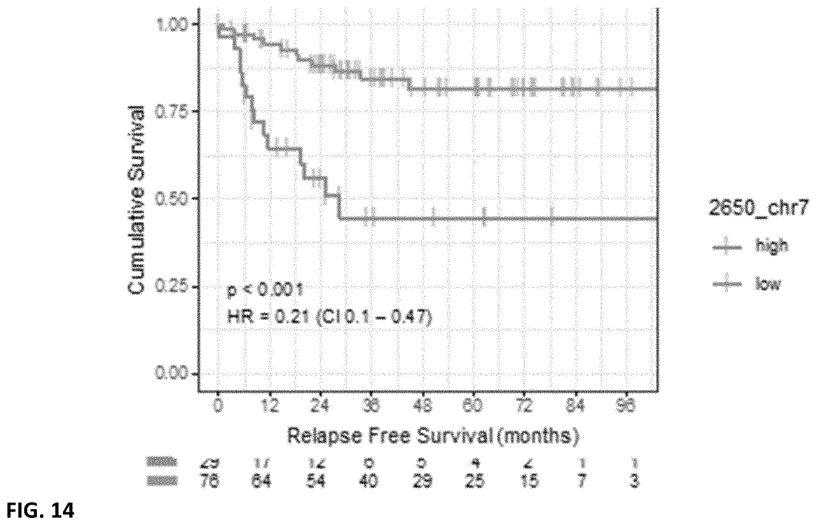

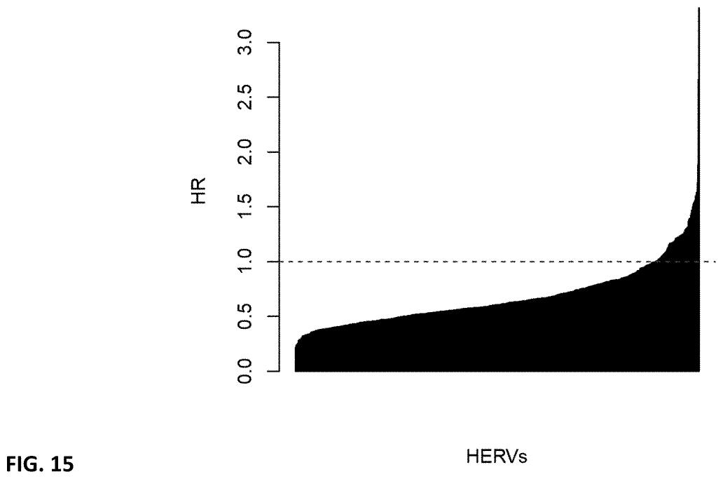

Without being limited by any particular theory, not all hERV have the same prognostic power. FIG. 15 shows the range of predictive power of hERVs. In some embodiments, “hERV 2650” located on chromosome 7 had the strongest predictive power (HR=0.21, P<0.001) (FIGS. 13 and 14). Without being limited by any particular theory, the location of hERV 2650 is not fixed to chromosome 7 and can move around in the genome. However, it is not the location of hERV 2650 but hERV 2650 itself that correlates with the predictive power.

Composite Score Based on Immune Cell Deconvolution and hERV Analyses

Some embodiments herein relate to human endogenous retroviral gene expression and immune cell infiltration as prognosis biomarkers in stage II/III colorectal cancer.

In some embodiments, tumor infiltrating lymphocytes (TILs) are closely related to hERV expression demonstrating immunogenicity of hERVs. Correlation with CD8 T cells is indicative of HERVs being immunogenic and very potent antigens.

Some embodiments are related to combining the expression profile of cell-type specific markers and expression profile of hERV/retro-transposon transactivation antigens to develop a composite score that may predict an outcome of anti-cancer treatment and overall survival or relapse free survival.

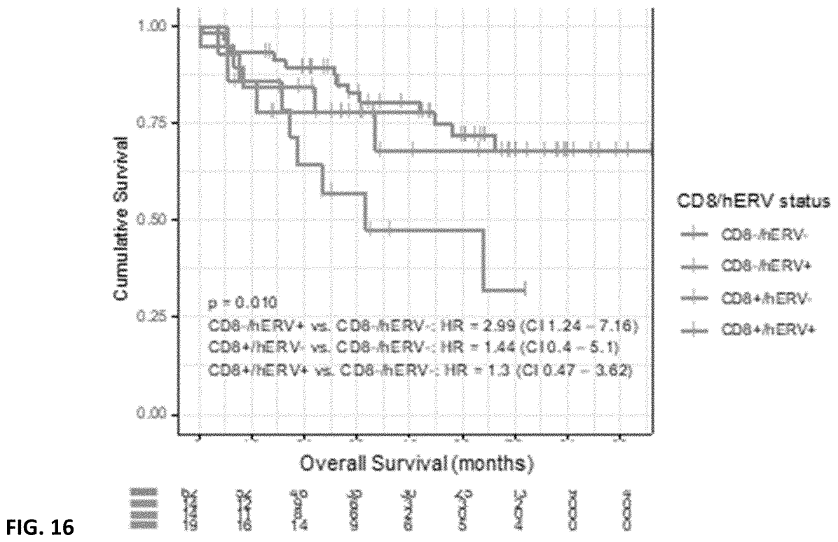

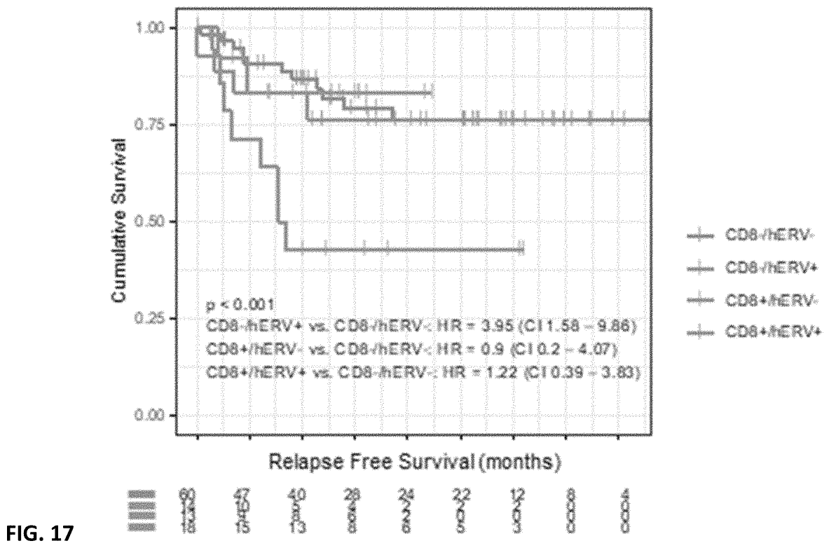

In some embodiments, the combined analysis serves as a prognostic indicator and enables segregation of the population based on overall survival (FIG. 16). In some embodiments, the CD8/HERV status was a status was a strong prognostic indicator. In some embodiments, a CD8−/hERV+ status was a strong prognostic indicator of worst overall survival (FIG. 16). Similar, in some embodiments, a CD8−/hERV+ status was a strong prognostic indicator of worst relapse free survival (FIG. 17). In some embodiments, a CD8+/hERV+ status correlated with metastasis and serves as a biomarker for metastasis requiring immediate treatment of these individuals.

As shown in FIG. 17, median OS (RFS) of CD8−/hERV+ subgroup was 29.8 (19.7) compared to 37.5 (32.8) for other subgroups (HR=4.4, log-rank P<0.001). In some embodiments, individuals with CD8+/hERV− subgroup have the best prognosis. In some embodiments, CD8 and hERV levels have synergic impact on survival. Without being limited by any particular theory, it is believed that hERV regulate cancer cell proliferation and survival through altering the expression of the c-Myc proto-oncogene.

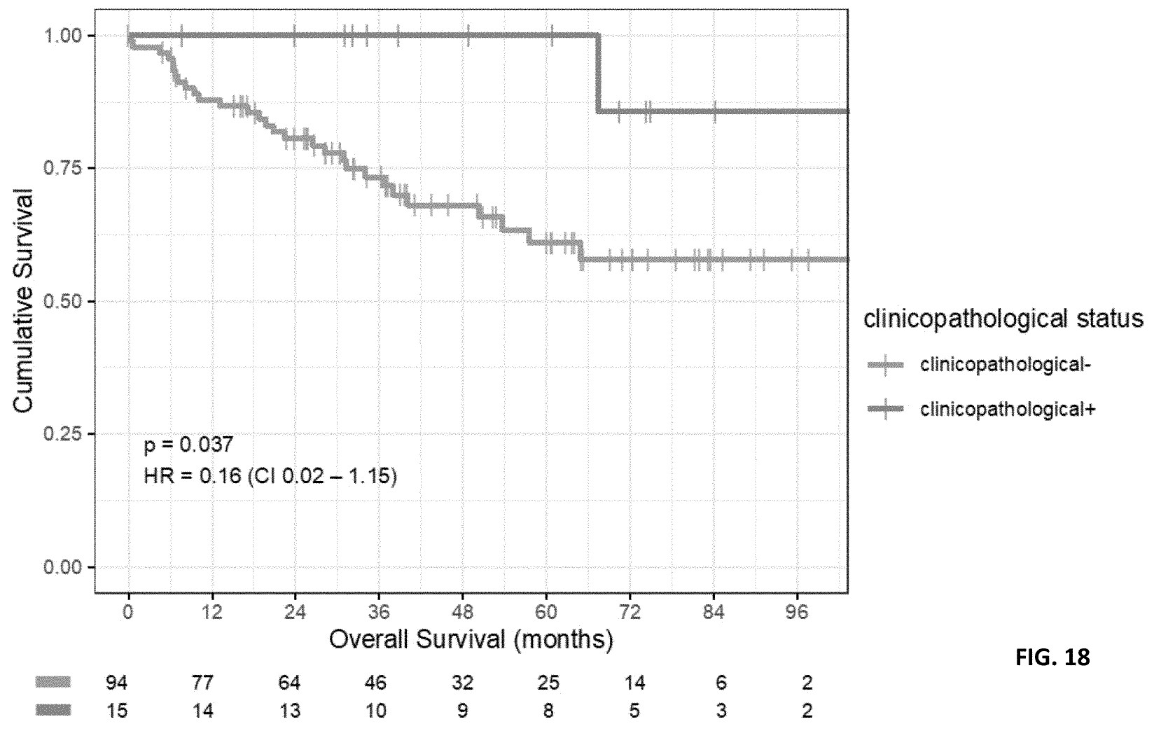

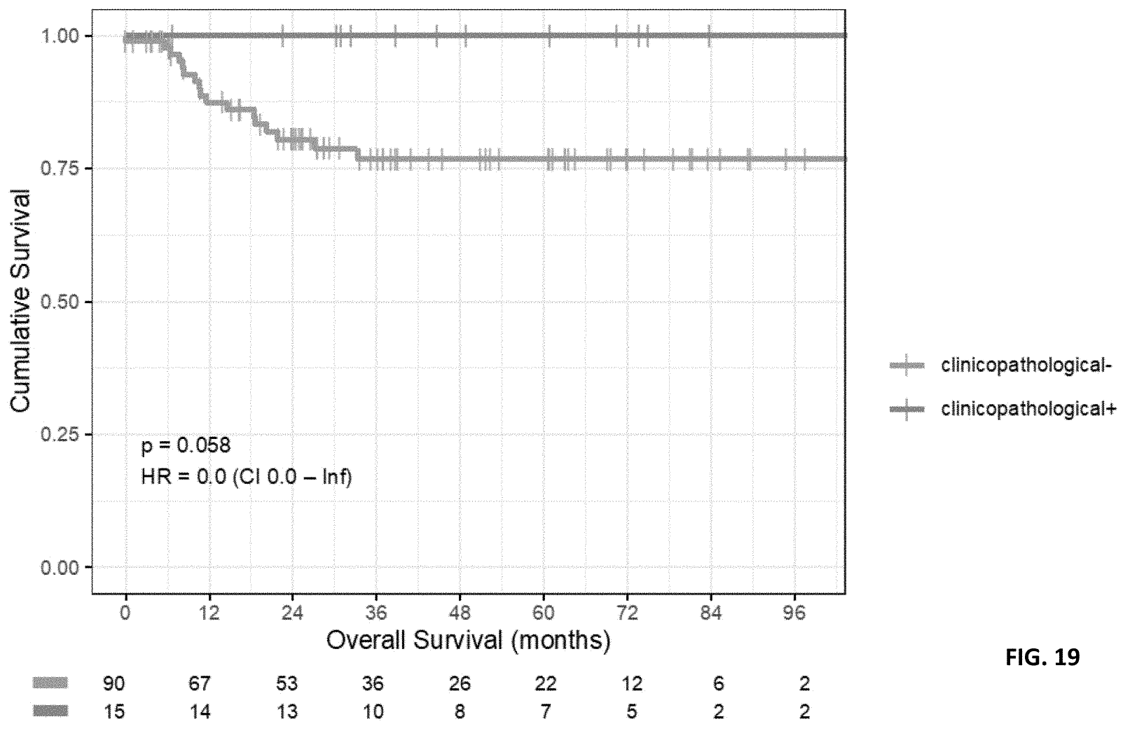

In some embodiments, one or more additional favorable and unfavorable traits/parameters including age of the individual, gender, stage of tumor, type of cancer, infection history of individual, cancer treatment regimens, sidedness, etc. can be included in the analysis to obtain a clinicopathological status and determine its correlation with overall survival and relapse free survival (FIGS. 18 and 19). In some embodiments, a clinicopathological negative status (i.e., presence of one or more favorable traits/absence of one or more unfavorable traits) is correlated with poor prognosis of overall survival (i.e., presence of more aggressive cancer and greater mortality), and a clinicopathological positive status (i.e., presence of one or more unfavorable traits/absence of one or more favorable traits) is correlated with better prognosis of overall survival (i.e., presence of less aggressive cancer and lower mortality) (FIGS. 18 and 19).

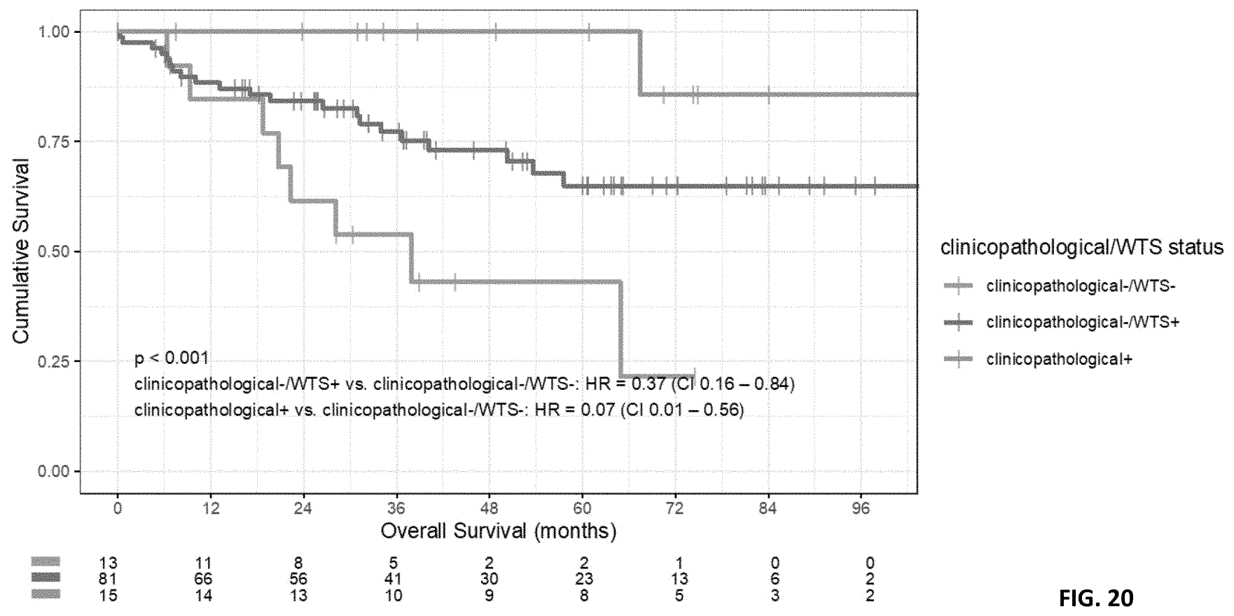

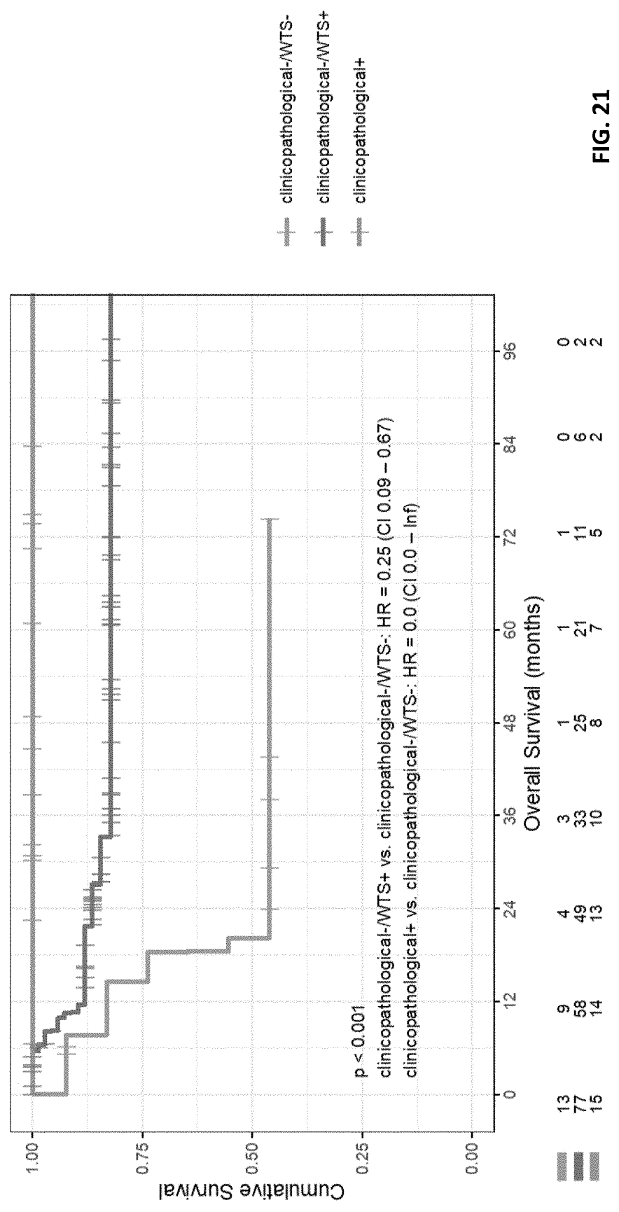

In some embodiments, combining clinicopathological negative status with the CD8−/hERV+ status (WTS− status) can deconvolve the poor clinicopathological group into two significantly distinct subgroups with different prognosis, i.e., clinicopathological negative/WTS− group and clinicopathological negative/WTS+ group (FIGS. 20 and 21). The clinicopathological negative/WTS− group had significantly worse prognosis as compared to the clinicopathological negative/WTS+ group.

Some embodiments relate to a method for accurate deconvolution of immune cells, measurements of HERVs as well as other biomarkers through WES/WTS sequencing and novel bioinformatics algorithms. Combining next-generation sequencing (NGS) based biomarkers with clinicopathological factors provides a better prediction of individual survival compared to clinicopathological biomarkers alone in CRC. Among several predictive biomarkers, CD8−/HERV+ strongly stratified individuals OS and RFS and revealed a previously unknown subset of CRC individuals with high risk of relapse, metastasis and death.

In some embodiments, the prognosis is better for some cancers versus other cancers. For example, as shown by the data in Table 1 below, the prognosis for right side CRC is better than the prognosis for left side CRC based on association between WES and WTS correlates.

TABLE 1
Cohort summary

| sidedness | right | left | other |
|---|---|---|---|
| Number of Patients | 73 | 33 | 7 |

| State | II | III | other |
|---|---|---|---|
| Number of Patients | 68 | 45 | 0 |

| Metastasis | no | yes |
|---|---|---|
| Number of Patients | 106 | 7 |

| MSI | MSH | MSS |
|---|---|---|
| Number of Patients | 56 | 57 |

In some embodiments, CRC is right sided. In some embodiments, CRC is left sided. Right sided CRC includes cancers of proximal colorectal cancers of the proximal two-thirds of the transverse colon, ascending colon, and cecum. Left sided CRCs include cancers of the distal colorectal cancers of the distal third of the transverse colon, splenic flexure, descending colon, sigmoid colon, and rectum).

### EXAMPLES

The following examples are non-limiting and other variants within the scope of the art also contemplated.

### Example 1

### Deconvolution Analysis Introduction

Fractional Recovery of Immune Cell Types In Oncology NGS (FRICTION) is a validated, quantitative immune cell type deconvolution tool for performing cell type deconvolution analysis. It uses a basis of labeled cell type signatures to predict the fraction of these signatures within an unknown mixture sample. To do so, it essentially models the mixture sample as a penalized linear combination of the basis signatures. This is done using a simple SVM-based model. The calculation is performed on a subset of the presumption of linear combination.

### Preparing RNA for Deconvolution

A ZIPPY pipeline was used for prepping the RNA-seq data. It performs the following processes: bcl2fastq, then STAR, then RSEM and also generates some statistics. Bcl2fastq is performed to demultiplex next generation sequencing output in a bcl format into appropriate FASTQ files. Then alignment of reads and gene expression quantification are performed using STAR (Dobin et al., Bioinformatics. 2013 Jan; 29(1): 15-21.) and RSEM (Li, B., Dewey, C.N. RSEM: accurate transcript quantification from RNA-Seq data with or without a reference genome. BMC Bioinformatics 12, 323 (2011)) third party software packages. FRICTION can take in .genes.results files from RSEM as input, or two-column (feature, value) files.

Once the pipeline has been run, samples are added to the sample manifest: mixture files.tsv contains a record of every deconvolution sample that has been run, and also is used by run deconvolution.py to ingest data. For each sample, the filename provided should be the “.genes.results” file generated from RSEM. The mixture files format is mostly straightforward, but it's worth analyzing the metadata column, which contains information about the samples. Metadata can be Boolean or categorical. One example of metadata may be: “tissue:melanoma;id:ff5;total”. This describes that sample was from melanoma, was in the 5th batch of fresh frozen samples run, and is total (as opposed to purified cell type) RNA. The run deconvolution.py interface provides useful tools, for example allowing the testing of all melanoma, all total RNA, all samples from experiment ff5, etc.

### Running Deconvolution Analysis

Deconvolution is performed using the script run_deconvolution.py. There are many helper functions within this script to ease the process. The main function within the script is run_id, which will run all the samples associated with that id in the mixture_files.tsv spreadsheet.

Below is an example of running all samples for one experiment using magnetic bead-bound bases:
run_id([‘ff5’], ‘racle.csv’, fname=‘racle_results_ff4_norm.csv’, normalize=True, plot=False, immune_pos_filters=[(‘type’, ‘magnetic’)])

Of particular note: run_id puts its results in a csv file with name equal to the fname argument.

Run deconvolution can also be run from the command line to deconvolve a single sample: python run_deconvolution.py (gene list file) (sample to deconvolve)

### Making a Function to Run the Deconvolver/rRunning the Raw Deconvolver

One alternative to run_id is to build a separate running function. That function may follow this pattern:
dcf=Deconvolutifier(normalize=normalize)
load_immune_bases(dcf, basis_list=[‘CD8’, ‘CD4’, ‘CD19’])
dcf. load genelist(gene list)
# load mixtures based on some criterion
samples=load_mixtures(dcf, positive_filters=filters)
dcf.filter_df(dcf.filter_genes)
results=[dcf.deconvolve(mixture, verbose=False) for mixture in samples]

### Gene Lists

The second argument to run_id is a gene list file. Currently, there are two gene lists that are used. The racle gene list may be found online.

The xgb_mad_v2 gene list was developed by using various heuristics together.

Deconvolution from ATAC-seq Data

Deconvolving the atac seq data was performed as follows:

Process ATAC seq data. Example atac zippy json:/home/awise/sngs/workflow/old_run_j_sons/atac9.json

For basis files, we merged them using another zippy script:/home/awise/sngs/workflow/merge_and_macs cd4.json

Now, there are files output from MACS with peaks. Now we want to reformat the macs files for deconvolution. This is done with/home/awise/atac/deconv/feature_select.py

Now we are ready to run deconvolution. However, there are many MACS peaks. We want to find a way of choosing the best of them. This is performed with atac_feature_select.py

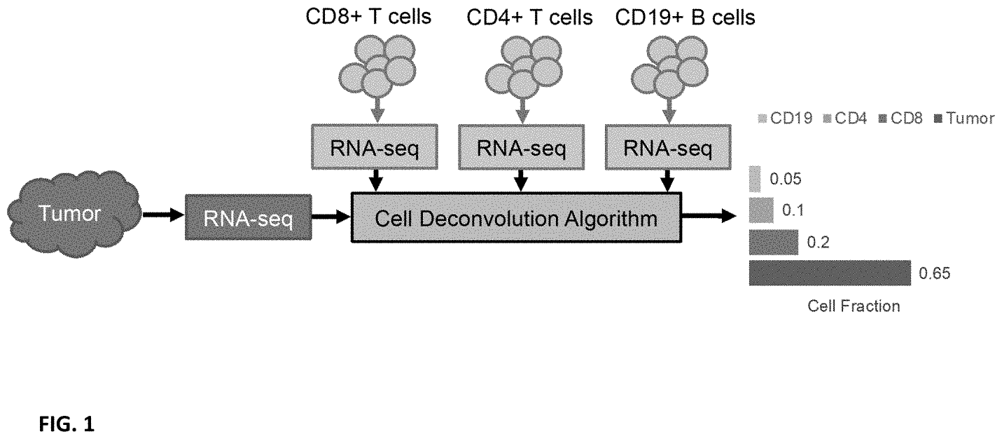

FRICTION is a new technique for performing immune cell type deconvolution from RNA-Seq data. FRICTION takes RNA-Seq measured from tumor samples, and uses a pre-built set of cell type signatures to predict the immune content of specific immune cell components (see FIG. 1).

FRICTION focuses on the selection and normalization of genes in ways that promote the detection of absolute cell fraction (i.e., the percentage of total cells) in contrast to other methods that focus on relative cell fraction (i.e., the percentage of immune cells) or statistical enrichment.

FRICTION works through a two-step process. First, a set of gene signatures are developed. In this experiment gene signatures were created using a set of purified cells, as well as an explicit set of background samples, from a variety of tissue types. Genes were then selected for deconvolution using three criteria:
Intra-to-inter class variance ratio. Genes are selected that are consistent within cell types, compared to global variation.
Maximum absolute deviation (MAD). Genes are selected that have large positive differences between at least one immune cell type and background.
Classification importance. For this process, an extreme gradient boosting classifier was built (Friedman, 2001) between each immune cell type and all background tissues and rank genes by their importance to the model.

Genes that scored well on the combination of these three measures were selected for performing deconvolution.

After the signatures are generated, FRICTION can be run from any human RNA-Seq sample. The deconvolution process may be run using a support vector regression based system inspired similar to that described by Newman et al., 2015. In contrast to Newman et al., but similar to Racle et al. 2017, we focused on the deconvolution of absolute cell fraction. This is enabled by our gene selection procedure, as well as our feature normalization that places each of our cell type signatures on the same scale.

FRICTION has been extensively evaluated using titration studies and sequenced tumors with orthogonal validation (IHC and flow cytometry).

Gene selection was performed using a set of magnetic-bead purified immune cell samples from three cell types (CD8+ T, CD4+ T and CD19+ B cells), with 6 CD8+ samples, 5 CD4+ samples and 4 CD19+ samples. Background tissue samples from ten tissue types (including lung, liver, colon, prostate and more) were used as controls. The resultant gene signature demonstrated both good separation between cell types as well as tight clustering of the background tissue types in a low-dimensional PCA representation (FIG. 2B).

FRICTION was evaluated using a series of titration experiments. In these experiments, known concentrations of CD8+, CD4+ and CD19+ primary cells were titrated into a variety of complex tissue backgrounds (primary individual tumors). Compared to simply looking at the correlation of marker genes and cell fraction, or using a simple hand-curated list of genes, FRICTION performs substantially better in terms of linear correlation, with median R² value >0.97 (Table 2). Further comparison to IHC stained lung and colon samples has demonstrated FRICTION's ability to distinguish high vs. low CD4+ T cell content in primary tumors (data not shown).

TABLE 2
Results of titration experiments.

| | | | | | CD-mini | | | | | |
|---|---|---|---|---|---|---|---|---|---|---|
| R² | % | TPM correlation | | | (8 gene panel) | | | FRICTION | | |
| values | Titration | CD8 | CD4 | CD19 | CD8 | CD4 | CD19 | CD8 | CD4 | CD19 |
| Colon #1 | 0-5% | 0.97 | 0.88 | 0.98 | 0.92 | 0.99 | 0.94 | 0.97 | 0.96 | 0.95 |
| Colon #2 | 0-5% | 0.98 | 0.88 | 0.99 | 0.97 | 0.99 | 0.98 | 0.96 | 0.96 | 0.98 |
| Colon #3 | 0-5% | 0.81 | 0.14 | 0.72 | 0.89 | 0.72 | 0.91 | 0.88 | 0.80 | 0.77 |
| Colon #4 | 0-5% | 0.99 | 0.56 | 0.99 | 0.91 | 0.99 | 0.93 | 0.94 | 0.96 | 0.98 |
| Kidney | 0-10% | 0.99 | 0.92 | 0.99 | 0.91 | 0.99 | 0.90 | 0.94 | 0.99 | 0.97 |
| Pancreas | 0-10% | 0.99 | 0.93 | 0.99 | 0.96 | 0.98 | 0.94 | 0.88 | 0.97 | 0.99 |
| Ovary | 0-10% | 0.97 | 0.00 | 0.99 | 0.85 | 0.94 | 0.89 | 0.87 | 0.99 | 0.97 |
| Rectum | 0-10% | 0.99 | 0.66 | 0.99 | 0.84 | 0.97 | 0.83 | 0.97 | 0.99 | 0.99 |
| Uterus | 0-5% | 0.68 | 0.26 | 0.99 | 0.65 | 0.82 | 0.72 | 0.93 | 0.98 | 0.99 |
| Esophagus | 0-5% | 0.99 | 0.45 | 0.99 | 0.77 | 0.98 | 0.86 | 0.90 | 0.99 | 0.97 |
| Thyroid | 0-5% | 0.98 | 0.05 | 0.98 | 0.91 | 0.91 | 0.81 | 0.93 | 0.98 | 0.99 |
| Bladder | 0-5% | 0.99 | 0.76 | 0.99 | 0.95 | 0.99 | 0.97 | 0.60 | 0.98 | 0.97 |

Three methods of deconvolution are compared, using raw correlation between normalized TPM (transcripts per million), a hand-curated panel of 8 CD genes and FRICTION's gene signatures. The ‘% titration’ column represents the dynamic range of the spike-in experiment (i.e., the maximum level of each immune cell type titrated).
Bold: P > 0.05
Italicized: P < 0.05 but R2 < 0.75

FRICTION has also been evaluated in comparison to 5 primary melanoma tumors quantified using flow cytometry (FIG. 2C). Concordance between predicted output and absolute cell fractions was found to be high for all three immune cell types predicted.

### Cell Deconvolution Including Gene Set Selection and Performance of Tissues

Primary immune cells were titrated into melanocyte cell background.

Training Set

1: CD8+ T cells (6 individuals)

2: CD4+ T cells (6 individuals)

3: CD19+ B cells (3 individuals)

Melanocyte Background

Sample #1: Melanocyte only

Sample #2: +0.6% CD4+, CD8+, and CD19+ cells

Sample #3: +3.3% CD4+, CD8+, and CD19+ cells

Sample #4: 11.8% CD4+, CD8+, and CD19+ cells

Titrations: Purified Immune Cells from Blood into Melanocytes

Mixture of immune cells (all same level)

Library Preparation: RNA Access (40 ng input)

Data are shown in FIG. 2D.

RNA from purified immune cells was titrated into RNA from total tissue (uterine tissue).

Training Set

1: CD8+ T cells (6 individuals)

2: CD4+ T cells (6 individuals)

3: CD19+ B cells (3 individuals)

Titrations: RNA of Purified Immune Cells from Blood Into RNA

RNA from purified immune cells from blood

Library Preparation: RNA Access (40 ng Input)

Data are shown in FIG. 2E and Table 3.

TABLE 3
Immune Cell RNA Spiked into Total RNA from Various Tissues (R2 values of linear correlation);

| | % | TPM | | | CM-mini | | | LM22 | | | Xgb | | |
|---|---|---|---|---|---|---|---|---|---|---|---|---|---|
| | Titration | CD8A | CD4 | CD19 | CD8 | CD4 | CD19 | CD8 | CD4 | CD19 | CD8 | CD4 | CD19 |
| Melanocyte | 0-11% | 0.93 | 0.92 | 0.96 | 0.01 | 0.97 | 0.76 | 0.73 | 0.94 | 0.80 | 0.98 | 0.99 | 0.99 |
| Colon | 0-5% | 0.97 | 0.88 | 0.98 | 0.92 | 0.99 | 0.94 | 0.94 | 0.95 | 0.98 | 0.97 | 0.96 | 0.95 |
| 12074 | | | | | | | | | | | | | |
| Colon | 0-5% | 0.98 | 0.88 | 0.99 | 0.97 | 0.99 | 0.98 | 0.98 | 0.91 | 0.98 | 0.96 | 0.96 | 0.98 |
| 11778 | | | | | | | | | | | | | |
| Colon | 0-5% | 0.81 | 0.14 | 0.72 | 0.89 | 0.72 | 0.91 | 0.81 | 0.45 | 0.32 | 0.88 | 0.80 | 0.77 |
| 11656 | | | | | | | | | | | | | |
| Colon | 0-5% | 0.99 | 0.56 | 0.99 | 0.91 | 0.99 | 0.93 | 0.97 | 0.82 | 0.92 | 0.94 | 0.96 | 0.98 |
| 12262 | | | | | | | | | | | | | |
| Kidney | 0-10% | 0.99 | 0.92 | 0.99 | 0.91 | 0.99 | 0.90 | 0.97 | 0.98 | 0.98 | 0.94 | 0.99 | 0.97 |
| Pancreas | 0-10% | 0.99 | 0.93 | 0.99 | 0.96 | 0.98 | 0.94 | 0.96 | 0.97 | 0.96 | 0.88 | 0.97 | 0.99 |
| Ovary | 0-10% | 0.97 | 0.00 | 0.99 | 0.85 | 0.94 | 0.89 | 0.94 | 0.98 | 0.98 | 0.87 | 0.99 | 0.97 |
| Rectum | 0-10% | 0.99 | 0.66 | 0.99 | 0.84 | 0.97 | 0.83 | 0.63 | 0.94 | 0.91 | 0.97 | 0.99 | 0.99 |
| Uterus | 0-5% | 0.68 | 0.26 | 0.99 | 0.65 | 0.82 | 0.72 | 0.70 | 0.79 | 0.86 | 0.93 | 0.98 | 0.99 |
| Esophagus | 0-5% | 0.99 | 0.45 | 0.99 | 0.77 | 0.98 | 0.86 | 0.83 | 0.99 | 0.90 | 0.90 | 0.99 | 0.97 |
| Thyroid | 0-5% | 0.98 | 0.05 | 0.98 | 0.91 | 0.91 | 0.81 | 0.81 | 0.82 | 0.66 | 0.93 | 0.98 | 0.99 |
| Bladder | 0-5% | 0.99 | 0.76 | 0.99 | 0.95 | 0.99 | 0.97 | 0.65 | 0.85 | 0.53 | 0.60 | 0.98 | 0.97 |
| Lung | | | | | | | | | | | | | |
| Liver | | | | | | | | | | | | | |

Bold: P value > 0.05,
Italicized: P value < 0.05; R2 < 0.75

FIG. 2F shows additional embodiments of examples of titration versus score. Linearity was observed from 0-5%. However, signature score tends to be lower than experimental spike-in (e.g., esophagus & bladder as extreme cases of CD8+ T cells), and slope is not the same across all spike-ins (e.g., liver CD8 versus CD4 slopes).

FIG. 2G shows additional embodiments of examples of titration versus score with technical replicates. The results were generally linear and showed good technical reproducibility. However, once again, signature score tends to be lower than experimental spike-in (e.g., rectum as extreme example), and slope is not the same across all spike-ins.

### Example 2

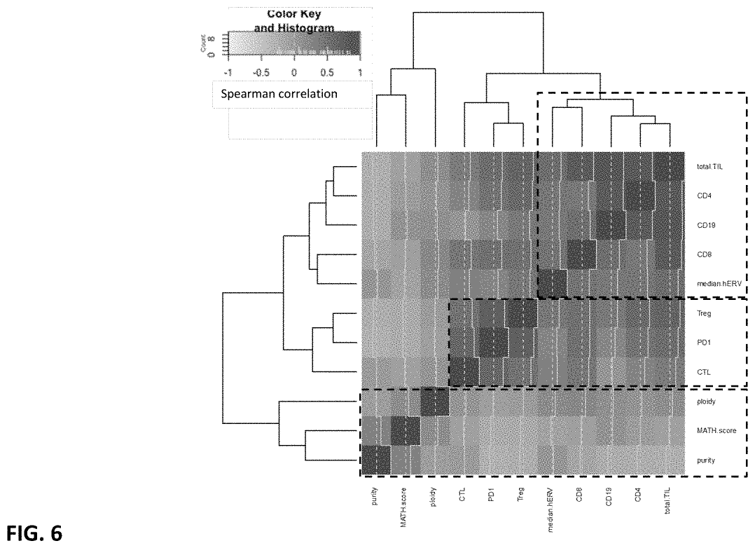

DNA and RNA were extracted from fresh-frozen tumor and matched normal tissues of 114 individuals with stage IUIII CRC with a 1-1 MSH/MSS ratio (measured by MSI-PCR) together with the clinical data including overall survival (OS), relapse free survival (RFS), sex, age, stage, sidedness, adjuvant treatment, and metastatic status. Whole Exome Sequencing (WES) and Whole Transcriptome Sequencing (WTS) libraries were generated using Illumina Nextera™ Flex for Enrichment, and TruSeq™ Stranded Total RNA library prep methods respectively, and sequenced on a NovaSeg™ 6000 system. FIG. 6 shows a heat map of the association between WES and WTS correlates.

Using an internally developed bioinformatics pipeline, various biomarkers such as human endogenous retroviral (HERV) gene expression, tumor infiltrating lymphocytes (TILs), microsatellite instability (MSI) status, tumor mutational burden (TMB), immune related gene expression were analyzed and the clinical significance of these signatures was evaluated.

### Example 3

Among clinicopathological factors, age, treatment, stage, and metastasis status were strong predictors of outcome. With WES and WTS derived biomarkers, MSI status together with HERV expression, CD8+ and CD19+ infiltration (as determined by a novel immune cell deconvolution-based method) were strong predictors. Interestingly, HERV expression and CD8+ cells have synergic impact on survival and median OS of CD8−/HERV+ subgroup is 29.8 compared to 37.5 for other subgroups (HR=4.4, log-rank P<0.001). Moreover, CD8−/HERV+ biomarker identified a more aggressive type of CRC that clinicopathological factors alone failed to uncover. Finally, a high correlation between the majority of detected HERV transcripts and TILs, was observed demonstrating the immunogenicity of these novel targets suggesting HERV expression as potential biomarker of response to immune-checkpoint inhibitors in CRC as well as other tumor types.

### Example 4

### HERV Quantification Algorithm

Provided herein is a HERV quantification process. A list of approximately 3000 genomic sequences belonging to human endogenous and exogenous retroviral genes was compiled. An alignment was performed of WTS-obtained reads using the custom index file based on this list appended to a hg19 human genome reference build. STAR and SALMON (Patro, et al., Nat Methods. 2017 Apr; 14(4): 417-419) third party alignment software was used and transcript quantification methods were employed using an optimized set of options. After quantification of these genes, library normalization was performed and to calculate median HERV values using the median normalized expression of all viral related genes for the sample.

### Hardware System

In some embodiments, the disclosed methods for determining a composite score are implemented in an application-specific hardware designed or programmed to compute the disclosed methods with higher efficiency than a general-purpose computer processor. For example, the process may be run using a general-purpose computer, or alternatively run using a field-programmable gate array (FPGA) or an application-specific integrated circuit (ASIC).

In some embodiments, one or more Application-Specific Integrated Circuits (ASICs) can be programmed to perform the functions of one or more of the respective methods described herein. ASICs include integrated circuits that include one or more programmable logic circuits that are similar to the FPGAs described herein in that the digital logic gates of the ASIC are programmable using a hardware description language such as VHDL. However, ASICs differ from FPGAs in that ASICs are programmable only once and cannot be dynamically reconfigured once programmed. Furthermore, aspects of the present disclosure are not limited to determining a composite score using FPGAs or ASICs. Instead, the main processing unit of any system performing the method may be implemented using one or more central processing units (CPUs), graphical processing units (GPUs), or any combination therefore.

In some implementations, the use of integrated circuits such as an FPGA, ASIC, CPU, GPU, or combination thereof, can include a single FPGA, a single ASIC, a single CPU, a single GPU, or any combination thereof. Alternatively, or in addition, the use of integrated circuits such as FPGA, ASIC, CPU, GPU, or combination thereof, can include multiple FPGAs, multiple ASICs, multiple CPUs, or multiple GPUs, or any combination thereof. The use of additional integrated circuits such as multiple FPGAs can reduce the amount of time it takes to perform additional analyses operations.

### Terminology

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g.,“a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of predicting the outcome of treating an individual suffering from cancer with an anti-cancer treatment, the method comprising:
obtaining RNA sequence expression data from a biopsy taken from an individual having cancer;
analyzing the RNA sequence expression data to determine if expression of cell-type specific markers in the biopsy are above a threshold value;
analyzing the RNA sequence expression data to determine if hERV/retro-transposon gene expression is found within the biopsy;
determining the cancer prognosis of the individual based on the threshold value and presence of the hERV/retro-transposon gene expression in the biopsy; and
combining the expression profile of cell-type specific markers and expression profile of hERV/retro-transposon transactivation antigens to predict an outcome of an anti-cancer treatment.

2. The method of claim 1, wherein the cancer comprises a tumor and the biopsy is a tumor biopsy.

3. The method of claim 1, wherein analyzing the RNA sequence expression data comprises performing a transcriptome sequence analysis of global human endogenous retrovirus (hERV)/retro-transposon transactivation.

4. The method of claim 1, wherein obtaining RNA sequence expression data comprises isolating total RNA from the cells, and performing next generation sequencing on the RNA sample to obtain the RNA sequence expression data.

5. The method of claim 1, wherein analyzing the RNA sequence expression data to determine if hERV/retro-transposon gene expression is found comprises measuring expression of the hERV 2650 gene located on chromosome 7.

6. The method of claim 1, wherein the cancer is selected from the group consisting of colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

7. The method of claim 1, wherein the anti-cancer treatment is selected from the group consisting of surgery, radiation therapy, chemotherapy, immunotherapy, targeted therapy, hormone therapy, stem cell transplant, cytokine therapy, gene therapy, cell therapy, phototherapy, thermotherapy, and sound therapy.

8. The method of claim 1, wherein the anti-cancer treatment comprises an anti-cancer chemotherapeutic selected from the group consisting of Cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

9. The method of claim 1, wherein the cell-type specific markers are selected from the group consisting of: human endogenous retroviral (HERV) gene expression markers, tumor infiltrating lymphocyte (TIL) markers, microsatellite instability (MSI) status markers, and tumor mutational burden (TMB) markers.

10. The method of claim 1, wherein the cell-type specific markers comprise markers associated with one or more of CD8+ T, CD4+ T, and CD19+ B cells.

11. The method of claim 1, wherein the hERV/retro-transposon gene expression level is calculated using a univariate analysis of hERV gene expression.

12. A method of obtaining a cellular signature of cells infiltrating a tumor, the method comprising:
obtaining a tumor;
isolating cells of the tumor;
isolating total RNA from the cells;
performing RNAseq to obtain RNA sequence expression data;
analyzing the RNA sequence expression data using a deconvolution algorithm to obtain an expression profile of cell-type specific markers; and
determining a fraction of a cell-type based on the expression profile of cell-type specific markers in the RNA sequence expression data.

13. The method of claim 12, further comprising:
comparing the expression profile of cell-type specific markers and/or the expression profile of hERV/retro-transposon transactivation antigens and/or the fraction of one or more immune cell types in the tumor to a predetermined threshold, and administering an immune checkpoint inhibitor therapy to a patient if the tumor obtained from said patient exhibits a fraction above the predetermined threshold.

14. The method of claim 12, wherein the cell-type specific markers comprise markers associated with one or more of CD8+ T, CD4+ T, and CD19+ B cells.

15. The method of claim 13, wherein the immune checkpoint inhibitor therapy comprises a checkpoint inhibitor selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

16. A method of obtaining a composite score of global human endogenous retrovirus (hERV)/retro-transposon transactivation, the method comprising:
obtaining a tumor;
isolating cells of the tumor;
isolating total RNA from the cells;
performing RNAseq to obtain RNA sequence expression data; and
analyzing the RNA sequence expression data to obtain an expression profile of hERV/retro-transposon transactivation antigens.

17. The method of claim 16, further comprising:
comparing the expression profile of cell-type specific markers and/or the expression profile of hERV/retro-transposon transactivation antigens and/or the fraction of one or more immune cell types in the tumor to a predetermined threshold, and administering an immune checkpoint inhibitor therapy to a patient if the tumor obtained from said patient exhibits a fraction above the predetermined threshold.

18. The method of claim 17, wherein the immune checkpoint inhibitor therapy comprises a checkpoint inhibitor selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).
